(19) United States
(12) Patent Application Publication
(10) Pub. No.: US 2003/0088277 A1
(43) Pub. Date: May 8, 2003

(54) Switched resistor defibrillation circuit

(76) Inventor: Alan H. Ostroff, San Clemente, CA (US)

Correspondence Address: BROBECK, PHLEGER & HARRISON LLP, 12390 EL CAMINO REAL, SAN DIEGO, CA, 92130, US

(21) Appl. No.: 10/011,565

(22) Filed: Nov. 5, 2001

(51) Int. Cl. A61N 1/39

(52) U.S. Cl. 607/5

(57) ABSTRACT

A defibrillator circuit for generating a rectangular waveform across a patient from capacitively stored energy and employing one or more capacitors initially charged to a common voltage and thereafter sequentially switchable with one or more resistors so as to raise the voltage supplied to an H-bridge circuit from a point of decay back to the common voltage.

### RELATED APPLICATIONS

The present invention may find application in systems such as are disclosed in the U.S. patent application entitled “SUBCUTANEOUS ONLY IMPLANTABLE CARDIOVERTER-DEFIBRILLATOR AND OPTIONAL PACER,” having Ser. No. 09/663,607, filed Sep. 18, 2000, pending, and U.S. patent application entitled “UNITARY SUBCUTANEOUS ONLY IMPLANTABLE CARDIOVERTER-DEFIBRILLATOR AND OPTIONAL PACER,” having Ser. No. 09/663,606, filed Sep. 18, 2000, pending, of which both applications are assigned to the assignee of the present application, and the disclosures of both applications are hereby incorporated by reference.

### FIELD OF THE INVENTION

The subject invention relates to electronic circuitry and particularly to circuitry having applications in defibrillating apparatus.

### BACKGROUND OF THE INVENTION

Defibrillation/cardioversion is a technique employed to counter arrhythmic heart conditions including some tachycardias in the atria and/or ventricles. Typically, electrodes are employed to stimulate the heart with electrical impulses or shocks, of a magnitude substantially greater than pulses used in cardiac pacing. Because current density is a key factor in both defibrillation and pacing, implantable devices may improve what is capable with the standard waveform where the current and voltage decay over the time of pulse deliver. Consequently, a waveform that maintains a constant current over the duration of delivery to the myocardium may improve defibrillation as well as pacing.

Defibrillation/cardioversion systems include body implantable electrodes that are connected to a hermetically sealed container housing the electronics, battery supply and capacitors. The entire system is referred to as implantable cardioverter/defibrillators (ICDs). The electrodes used in ICDs can be in the form of patches applied directly to epicardial tissue, or, more commonly, are on the distal regions of small cylindrical insulated catheters that typically enter the subclavian venous system, pass through the superior vena cava and, into one or more endocardial areas of the heart. Such electrode systems are called intravascular or transvenous electrodes. U.S. Pat. Nos. 4,603,705, 4,693,253, 4,944,300, 5,105,810, the disclosures of which are all incorporated herein by reference, disclose intravascular or transvenous electrodes, employed either alone, in combination with other intravascular or transvenous electrodes, or in combination with an epicardial patch or subcutaneous electrodes. Compliant epicardial defibrillator electrodes are disclosed in U.S. Pat. Nos. 4,567,900 and 5,618,287, the disclosures of which are incorporated herein by reference. A sensing epicardial electrode configuration is disclosed in U.S. Pat. No. 5,476,503, the disclosure of which is incorporated herein by reference.

In addition to epicardial and transvenous electrodes, subcutaneous electrode systems have also been developed. For example, U.S. Pat. Nos. 5,342,407 and 5,603,732, the disclosures of which are incorporated herein by reference, teach the use of a pulse monitor/generator surgically implanted into the abdomen and subcutaneous electrodes implanted in the thorax. This system is far more complicated to use than current ICD systems using transvenous lead systems together with an active can electrode and therefore it has no practical use. It has in fact never been used because of the surgical difficulty of applying such a device (3 incisions), the impractical abdominal location of the generator and the electrically poor sensing and defibrillation aspects of such a system.

Recent efforts to improve the efficiency of ICDs have led manufacturers to produce ICDs which are small enough to be implanted in the pectoral region. In addition, advances in circuit design have enabled the housing of the ICD to form a subcutaneous electrode. Some examples of ICDs in which the housing of the ICD serves as an optional additional electrode are described in U.S. Pat. Nos. 5,133,353, 5,261,400, 5,620,477, and 5,658,321 the disclosures of which are incorporated herein by reference. ICDs are now an established therapy for the management of life threatening cardiac rhythm disorders, primarily ventricular fibrillation (V-Fib). ICDs are very effective at treating V-Fib, but are therapies that still require significant surgery.

As ICD therapy becomes more prophylactic in nature and used in progressively less ill individuals, especially children at risk of cardiac arrest, the requirement of ICD therapy to use intravenous catheters and transvenous leads is an impediment to very long term management as most individuals will begin to develop complications related to lead system malfunction sometime in the 5-10 year time frame, often earlier. In addition, chronic transvenous lead systems, their reimplantation and removals, can damage major cardiovascular venous systems and the tricuspid valve, as well as result in life threatening perforations of the great vessels and heart. Consequently, use of transvenous lead systems, despite their many advantages, are not without their chronic patient management limitations in those with life expectancies of >5 years. The problem of lead complications is even greater in children where body growth can substantially alter transvenous lead function and lead to additional cardiovascular problems and revisions. Moreover, transvenous ICD systems also increase cost and require specialized interventional rooms and equipment as well as special skill for insertion. These systems are typically implanted by cardiac electrophysiologists who have had a great deal of extra training.

In addition to the background related to ICD therapy, the present invention requires a brief understanding of a related therapy, the automatic external defibrillator (AED). AEDs employ the use of cutaneous patch electrodes, rather than implantable lead systems, to effect defibrillation under the direction of a bystander user who treats the patient suffering from V-Fib with a portable device containing the necessary electronics and power supply that allows defibrillation. AEDs can be nearly as effective as an ICD for defibrillation if applied to the victim of ventricular fibrillation promptly, i.e., within 2 to 3 minutes of the onset of the ventricular fibrillation.

AED therapy has great appeal as a tool for diminishing the risk of death in public venues such as in air flight. However, an AED must be used by another individual, not the person suffering from the potential fatal rhythm. It is more of a public health tool than a patient-specific tool like an ICD. Because >75% of cardiac arrests occur in the home, and over half occur in the bedroom, patients at risk of cardiac arrest are often alone or asleep and can not be helped in time with an AED. Moreover, its success depends to a reasonable degree on an acceptable level of skill and calm by the bystander user.

What is needed therefore, especially for children and for prophylactic long term use for those at risk of cardiac arrest, is a combination of the two forms of therapy which would provide prompt and near-certain defibrillation, like an ICD, but without the long-term adverse sequelae of a transvenous lead system while simultaneously using most of the simpler and lower cost technology of an AED. What is also needed is a cardioverter/defibrillator that is of simple design and can be comfortably implanted in a patient for many years.

### SUMMARY

A defibrillator circuit for generating a rectangular waveform across a patient from capacitively stored energy and employing one or more capacitors initially charged to a common voltage and thereafter sequentially switchable with one or more resistors so as to raise the voltage supplied to an H-bridge circuit from a point of decay back to the common voltage.

### BRIEF DESCRIPTION OF THE DRAWINGS

For a better understanding of the invention, reference is now made to the drawings where like numerals represent similar objects throughout the figures and wherein:

FIG. 1 is an electrical circuit schematic of an illustrative embodiment of the invention;

FIG. 2 is a waveform diagram illustrative of operation of the circuit of FIG. 1; and

FIG. 3 is a waveform diagram illustrative of operation of the circuit of FIG. 1.

### DETAILED DESCRIPTION OF ILLUSTRATIVE EMBODIMENTS

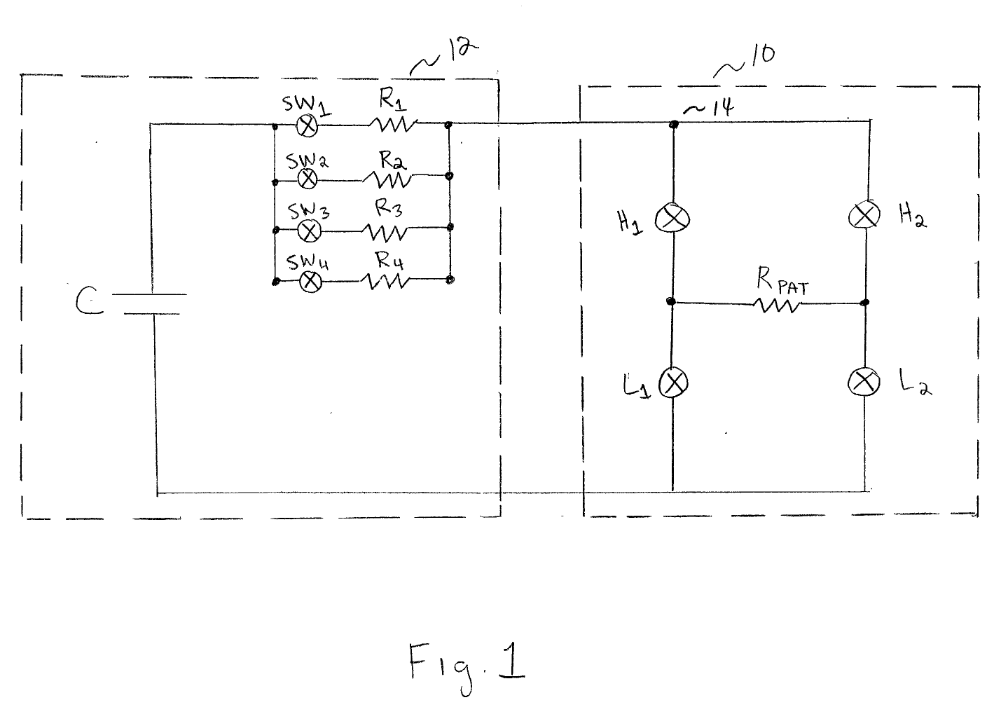

An illustrative embodiment is shown in FIG. 1. The illustrative embodiment includes an H bridge circuit 10 and a drive circuit 12 for supplying voltage or energy to the H bridge circuit 10.

The H bridge circuit 10 may be of conventional form, including first and second high side switches H₁, H₂ and first and second low side switches L₁, L₂. The switches H₁, H₂, L₁, L₂ may be manipulated to appropriately and selectively apply a voltage present at junction 14 across a patient indicated by a patient resistance R_{PAT}.

In an embodiment, the drive circuit 12 of FIG. 1 includes a plurality of electrical resistance devices in the illustrative form of four resistors R₁, R₂, R₃, R₄. One end of the resistors R₁, R₂, R₃, R₄ is connected to junction 14. The other end of the resistors R₁, R₂, R₃, R₄ is connected to one end of switches SW₁, SW₂, SW₃, Sw₄, respectively. The other end of each of the switches SW₁, SW₂, SW₃, Sw₄, is connected to a high voltage capacitor V_{C} having a capacitance C. In an embodiment, the high voltage capacitor C provides a source of D.C. voltage of approximately 350 volts to approximately 3500 volts.

The resistors R₁, R₂, R₃, R₄ are switchable via respective switches SW₁, SW₂, SW₃, SW₄ to establish or remove an electrical connection between the high voltage capacitor V_{C} and the junction 14. In an embodiment, the values of the resistors R₁, R₂, R₃, R₄ are determined such that R₁>R₂>R₃>R₄. Typically, the value of R₄ is approximately zero (i.e., a short circuit).

In illustrative operation of the circuit of FIG. 1, switch SW₁, the first high side switch H₁, the second low side switch L₂ are closed, while the second high side switch H₂ and the first low side switch L₁ are open, thereby connecting the voltage on the high voltage capacitor V_{C} across the resistor R₁ and the patient resistance R_{PAT}.

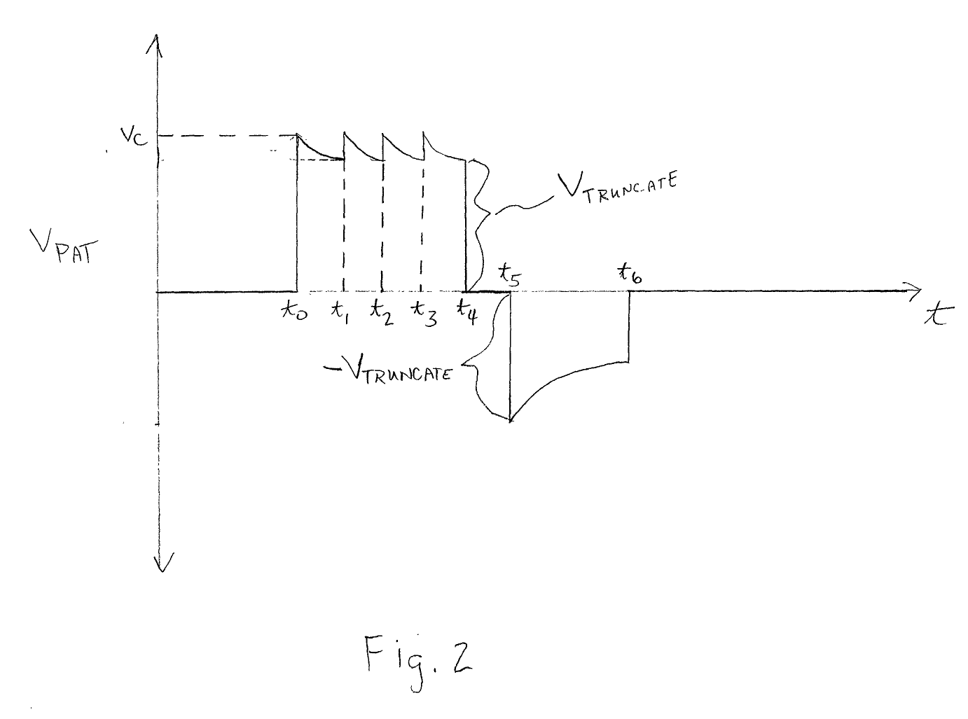

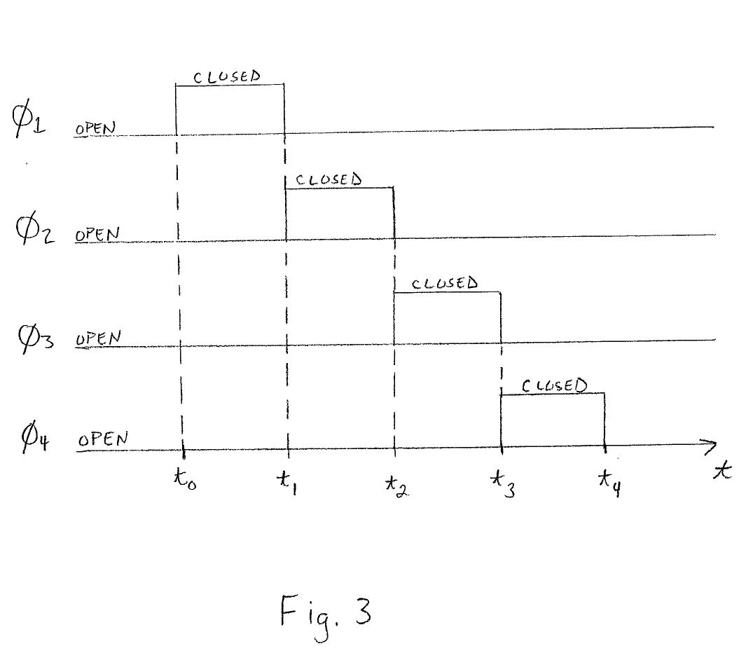

As shown in FIG. 2, the voltage across the patient is initially V_{PAT} and decays with a time constant proportional to (R₁+R_{PAT}) (C) for a selected time period up to a point in time denoted t₁ in FIG. 2. At time t₁, a switching signal Φ₂ (FIG. 3) is activated to close switch SW₂. The value of R₂ is chosen so that the patient voltage V_{PAT} initially rises back up to its original value and then begins to decay with a time constant proportional to (R₁∥R₂+R_{PAT}) (C).

At a selected time t₂, a switching signal Φ₃ (FIG. 3) is activated to close switch SW₃. The value of R₃ is chosen so that the patient voltage V_{PAT} again rises back up to its original value and then begins to decay with a time constant proportional to (R₁∥R₂∥R₃+R_{PAT}) (C).

At a selected time t₃, a switching signal Φ₄ (FIG. 3) is activated to close switch SW₄. Because the value of R₄ is approximately zero, the patient voltage V_{PAT} once again rises back up to its original value and thereafter decaying with a time constant proportional to (R₁∥R₂∥R₃∥R₄+R_{PAT}) (C). However, because R₄ is typically zero, the voltage VPAT decays with a time constant proportional to (R_{PAT}) (C). Finally, at time t₄, the switches H₁, L₂ are opened, thereby terminating the first phase of the waveform at a voltage V_{TRUNCATE} as shown in FIG. 2.

If desired, these switches H₁, L₂ may then be closed to produce a conventional second phase 19 of a biphasic waveform. As shown in FIG. 2, this waveform drops to a voltage V_{TRUNCATE} at time t₅ and then decays with a time constant determined by the patient resistance R_{PAT}. Finally, the conventional second phase 19 is truncated at time t₆.

In an embodiment, typical values for resistors R₁, R₂, R₃, R₄ typical values may be approximately 50, 25, 10, and 0 ohms, respectively. In addition, typical values for times t₁, t₂, t₃, t₄, t₅, t₆ are approximately 1, 2, 3, 4, 5, and 9 milliseconds, respectively (assuming time t₀ is zero milliseconds).

While the present invention has been described above in terms of specific embodiments, it is to be understood that the invention is not limited to the disclosed embodiments. On the contrary, the present invention is intended to cover various modifications and equivalent methods and structures included within the spirit and scope of the appended claims.

What is claimed is:

1. An apparatus comprising:
an H-bridge circuit adapted to be connected to a patient; and
a drive circuit connected to the H-bridge circuit and including an energy storage device, a plurality of electrical resistance devices, and a plurality of switches, the switches enabling each electrical resistance device to be sequentially connected to the energy storage device to supply a drive voltage to the H-bridge circuit.

2. The apparatus of claim 1, wherein the energy storage device comprises a capacitor.

3. The apparatus of claim 1, wherein each of the electrical resistance devices comprises a resistor.

4. The apparatus of claim 3, wherein one or more resistors are employed.

5. The apparatus of claim 3, wherein the time of switching of each of the plurality of electrical resistance devices is selected such that an approximation of a rectangular voltage wave is applied across the patient.

6. An apparatus of claim 5, wherein after application of the approximation of a rectangular voltage, a plurality of switches are controlled so as to produce a phase of a biphasic waveform that is opposite in polarity to the rectangular voltage.

7. An apparatus comprising:
first and second switches adapted to be connected across a patient resistance and activatable when so connected to deliver a current to the patient in response to application of a voltage to the first and second switches; and
means including a plurality of electrical resistance means selectably switchable for providing an approximation of a rectangular voltage waveform to the first and second switches.

8. The apparatus of claim 7, wherein the first and second switches comprise part of an H-bridge circuit.

9. The apparatus of claim 7, wherein the waveform rises to a first voltage level, decays for a selected time interval and thereafter experiences a second rise to the first voltage level and decays for a second selected time interval.

10. The apparatus of claim 9, wherein the plurality of electrical resistance means includes one or more resistors.

11. The apparatus of claim 10, wherein the second rise and second decay are caused by switching of a second resistor into the electrical path of the current.

12. The apparatus of claim 11, wherein the means includes a plurality of switches selectively activated to switch the first and second resistors.

13. The apparatus of claim 10, wherein the second decay is a function of a time constant including a value of the second resistor.

14. The apparatus of claim 9, wherein the means includes one or more resistors and a plurality of switches permitting the resistors to be selectively coupled into the current.

15. The apparatus of claim 14, wherein the resistors are selectively coupled so as to create a plurality of decays proportional to the respective values of the one or more resistors.

16. The apparatus of claim 10, further comprising one or more capacitors, each switchable into a configuration with the one or more resistors.

17. The apparatus of claim 7, wherein the first and second switches form part of an H-bridge circuit.

18. A method of generating a drive signal for use in delivering a defibrillating signal to a patient comprising the steps of:
charging one or more capacitors to a common voltage;
applying the voltage on the one or more capacitors to create the drive signal; and
selectively connecting at least one of a plurality of resistors in series with the one or more capacitors.

19. The method of claim 18, wherein each of the remaining resistors is selectively coupled into a series configuration with the one or more capacitors.

20. The method of claim 18, wherein each of the resistors is sequentially coupled into the series configuration.

21. The method of claim 19, wherein the timing of coupling of each successive resistor into the series configuration is selected such that the drive signal approximates a rectangular pulse.
